(19) United States
(12) Patent Application Publication
(10) Pub. No.: US 2017/0215785 A1
(43) Pub. Date: Aug. 3, 2017

(54) TEMPORAL DEPENDENT LOCATION VALIDATION METHOD AND PERSONAL COMPLIANCE VALIDATION SYSTEM

(71) Applicant: James F. Marino, La Jolla, CA (US)

(72) Inventor: James F. Marino, La Jolla, CA (US)

(21) Appl. No.: 15/500,811

(22) PCT Filed: Jul. 30, 2015
(86) PCT No.: PCT/US2015/042927

§ 371 Date: Jan. 31, 2017

Related U.S. Application Data
(60) Provisional application No. 62/073,248, filed on Oct. 31, 2014.
(60) Provisional application No. 62/032,157, filed on Aug. 1, 2014.

(51) Int. Cl. A61B 5/18; A61B 5/11; A61B 5/00; A61B 5/1172; A61B 5/024

(52) U.S. Cl. CPC A61B 5/18; A61B 5/1172; A61B 5/024; A61B 5/6824; A61B 5/6831; A61B 5/742; A61B 5/1112

(57) ABSTRACT

Methods and systems are adapted for monitoring, verifying, and/or validating an individual's sensorial responsiveness, cognitive function, as well as compliance with mandatory rest intervals associated with a task, such as commercial truck driving or aircraft piloting. The system includes a sensorial stimulus and response assessment method and related devices, as well as a personal validation device that is specific to an individual and the interval monitored.

### REFERENCE TO PRIORITY DOCUMENTS

This application claims priority to the following co-pending U.S. patent applications: (1) U.S. Provisional Patent Application Ser. No. 62/032,157 entitled “Temporal Dependent Location Validation Method and Personal Compliance Validation System” and filed on Aug. 1, 2014; and (2) U.S. Provisional Patent Application Ser. No. 62/073,248 entitled “Temporal Dependent Location Validation Method and Sensorial Arousal/Cognitive Functionality Determination With Personal Compliance Validation System” and filed on Oct. 31, 2014. Priority to the aforementioned filing dates is claimed and the provisional patent applications are incorporated herein by reference in their entirety.

### BACKGROUND

There are instances in which individual compliance with specific corporate, industry, or governmental work requirements necessitate an understanding and/or monitoring of the individual's location at specific times during a specific period of time as well as their state of arousal/cognitive functionality. For example, there are performance requirements for alertness and cognitive functionality, as well as requirements for mandatory rest intervals associated with the work hours of commercial pilots and commercial truck drivers.

Current efforts to ensure compliance with some of these requirements range from voluntary self-reporting, to third party oversight, to expensive localization technologies (e.g. “home detention” bracelets with GPS monitors); as well as various means of detecting drowsiness or sleep deprivation performance degradation, such as head posture detection, eyelid position, and lane deviation detection. Unfortunately, current compliance methods can be retrospective, expensive, non-specific, and unreliable. Some compliance methods rely completely on voluntary self-reporting, which is dependent on the diligence of individuals who may fail to comply with the reporting requirements. Perhaps most important, these compliance reporting methods are retrospective deterrent means rather than providing for active prophylactic hazard intervention. Voluntary self-reporting compliance methods are subject to erroneous and/or fraudulent reporting and are therefore of limited reliability or value. Other compliance processes, such as third party oversights, have been shown to be cumbersome, expensive, and at times, ineffective.

In a Journal of Public Health Policy article, “Long Hours and Fatigue: A Survey of Tractor-Trailer Drivers,” investigators interviewed semi-trailer drivers at inspections stations and truck stops in Connecticut, Florida, Oklahoma, and Oregon. Two thirds of the interviewed drivers reported falsifying their logbook entries in an effort to misrepresent the extent of sleep deprivation they experienced during their driving activities. Drivers are twice as likely to have an accident if they are behind the wheel for eight hours or more, compared to well-rested drivers.

In view of the foregoing, there are needs for reliable, low cost, and portable methods and devices for monitoring and ensuring compliance with the arousal state, cognitive functionality and temporal work requirements of a specific individual during a specific period of time and/or activity. The inventions and methods described herein endeavor to provide practical means of assessing, monitoring, documenting, and intervening with respect to these needs.

### SUMMARY

Disclosed are methods and systems for monitoring, verifying, and/or validating an individual's sensorial responsiveness, cognitive function, as well as compliance with mandatory rest intervals associated with a task such as commercial truck driving or aircraft piloting. The system(s) includes a sensorial stimulus and response assessment method and related devices, as well as a personal validation device that is specific to an individual and the interval monitored. The device is configured to be secured to the individual such that the device cannot be removed from the individual without being identifiably altered, reset, or destroyed. In an embodiment, the personal validation device is a personal time and position data logger with associated unique encoded alphanumeric for the period of monitoring and the specific monitored individual. The validation device can interface with a confirmatory biometric recording device, such as a scanned or recorded anatomic identifying image or voice recognition capability, at the beginning end of the monitoring interval.

In one aspect, a unique, randomly generated alphanumeric sequence is associated with a biometric (for example, at least one of a palm print, digital print, voice recognition, voice recognized identification of the monitored party, etc.), which unique, randomly generated alphanumeric sequence is generated and recorded at the beginning of a period of monitoring and generated and recorded at the end of a period of monitoring for a monitored party. In an embodiment, the period of monitoring is initiated and terminated with the fastening and unfastening of a global positioning system (GPS)/random alphanumeric generator device (a personal monitoring device), which may be in the form of a wrist band or watch for example and which is sometimes referred to as a validation device or personal monitoring device. The personal monitoring device communicates with a smart phone or other data transmitting device. When the monitored party commences a period of monitoring, the monitored party secures the personal monitoring device to his or her wrist while simultaneously transmitting a displayed, randomly generated alphanumeric sequence and biometric (for example, “voice print” with password, palm print via a photograph and confirmatory wireless communication from the “watch” to the phone) over the transmitting device (such as a smart phone) to a monitoring site or agent. At end of the monitoring interval, a similar transmission occurs, before the personal monitoring device is dissociated from the monitored individual's wrist.

GPS positional data and periodic cognitive assessment information (such as testing of arousal state and cognitive capacity during the monitoring period) is transmitted and stored, on the device and at a remote location. This ensures that the monitored party is in appropriate locations and properly alert and capable of responding to interval cognitive sampling tests, during and only during the period of monitoring. In addition to cognitive testing, a recording of an individual's physiological parameter, such as the individual's pulse rate is monitored to indirectly reflect periods of compulsory rest. The monitored party is always free to interrupt or end the monitoring at any time and the monitoring is not active during non-monitored intervals (e.g. off shift). The monitoring is only done during appropriate intervals and always with the cooperation and immediate volitional consent of the monitored party. The monitoring may be performed as a previously consented to and required condition of employment for example. For example, no monitoring is performed without a formal pre-existing contractual understanding between the monitored party and a monitoring party or entity.

If the monitored party elects to discontinue monitoring during a period of normally required and/or desired monitored activity, monitored party should have a proper explanation for prematurely terminating the monitoring interval, or invite reasonable challenge to their motives for electing to go “off-monitoring”. Interruptions of monitoring for breaks, lunch, etc., can be easily accommodated.

In another aspect, the is disclosed a method of monitoring an individual, comprising: securing a validation device to a monitored individual; generating a unique identifying sequence upon securing the validation device to the individual; making a first record of the alphanumeric sequence on the monitored individual showing an identifying feature of the validation device and a biometric associated with the individual; electronically transmitting the first record to a logging center site that stores the record with a time and date stamp; after an interval of time, making a second record of the validation device on the monitored individual showing the alphanumeric sequence, an identifying feature of the validation device and a biometrics; and electronically transmitting the second record to the logging center site that stores the record with a time and date stamp.

Other features and advantages should be apparent from the following description of various embodiments, which illustrate, by way of example, the principles of the disclosure.

### BRIEF DESCRIPTION OF THE DRAWINGS

These and other aspects will now be described in detail with reference to the following drawings.

FIG. 1 shows a schematic representation of a system for monitoring and verifying compliance with temporal and/or location requirements for an individual.

FIG. 2 shows an exemplary compliance device in the form of a frangible wrist band.

FIG. 3 shows an alternate embodiment.

### DETAILED DESCRIPTION

Before the present subject matter is further described, it is to be understood that this subject matter described herein is not limited to particular embodiments described, as such may of course vary. It is also to be understood that the terminology used herein is for the purpose of describing a particular embodiment or embodiments only, and is not intended to be limiting. Unless defined otherwise, all technical terms used herein have the same meaning as commonly understood by one skilled in the art to which this subject matter belongs.

Disclosed are methods and systems for monitoring, verifying, and/or validating an individual's sensorial responsiveness, cognitive function, as well as compliance with mandatory rest intervals associated with a task such as commercial truck driving or aircraft piloting. The system(s) includes a sensorial stimulus and response assessment method and related devices, as well as a personal validation device that is specific to an individual and the interval monitored. The device is configured to be secured to the individual such that the device cannot be removed from the individual without being identifiably altered, reset, or destroyed. In an embodiment, the personal validation device is a personal time and position data logger with associated unique encoded alphanumeric for the period of monitoring and the specific monitored individual. The validation device can interface with a confirmatory biometric recording device, such as a scanned or recorded anatomic identifying image or voice recognition capability, at the beginning end of the monitoring interval.

The validation device includes a time and positional data logger, such as a global positioning system (GPS) that monitors the location of the individual as a function of time. The validation device also includes registration of unique identifiers, for the monitored individual and the specific period of monitored time. At least one biometric identifier is recorded or registered with the initiation and termination of a specific recording interval. An anatomic or physiologic characteristic that is unique or relatively unique to the monitored individual, such as a palm print, finger print, or voice inflection, is associated with the individual and scanned, imaged, or ascertained with the validation process at the beginning and end of each monitored interval and/or with the verification of a valid response to sensorial and/or cognitive monitoring activities. Pursuant to a method of operation, the wearer of the validation device secures it to his self and a communication is established with a verification or logging center to verify the individual's compliance, as described more fully below.

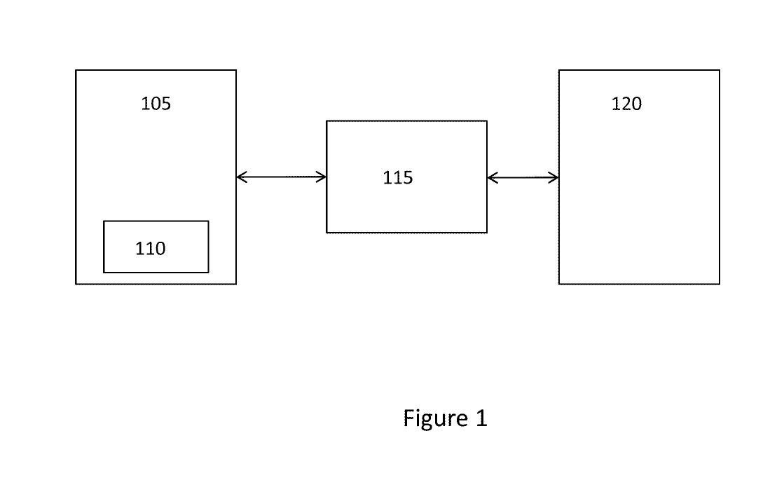

FIG. 1 shows a schematic representation of an exemplary embodiment of a personal compliance validation system that can be used to monitor, verify, and/or validate an individual's compliance with mandatory rest intervals associate with a task. The system is described herein in the context of monitoring a commercial truck driver's compliance with mandatory rest intervals during commercial driving. It should be appreciated that this is an example and that uses in other contexts are within the scope of this disclosure.

With reference still to FIG. 1, an individual 105 such as a truck driver is outfitted or equipped with a personal validation device 110, which is a device that is secured to the individual as described further below. The validation device 110 includes one or more identifiers that associate the device with the individual. A recording and/or communication device, such as an electronic scanner 115 and time and position data logger, can be used to record that the specific individual and the attached validation device are at a certain location at a certain time. The scanner 115 can then send recorded data to a logging center 120 that is used to verify the individual's compliance. This process is described in more detail below.

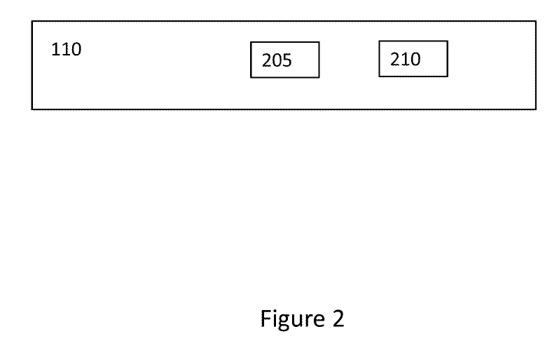

FIG. 2 shows an example of a validation device 110, which is a device that can be secured to the individual to be monitored. In an example embodiment, the validation device 110 is a wristband that is secured to a wearer's wrist in a frangible manner. That is, the validation device 110 (such as a wristband) is securable around a wearer's wrist and can only be removed by breaking the wristband into fragments. The wristband may include a frangible integrity seal that remains intact until the wristband is removed from the wearer's wrist. The wristband 105 includes at least one unique identifying data encoded inscription, such as a one or two-dimensional bar code 205. A data logger, such as a GPS/time data logger 210, having a unique and registered identifying alphanumeric sequence, is visibly embossed on the wristband, such as on volar surface of the wrist band.

The unique identifier can be generated by an identifying sequence generator, such a microprocessor that is programmed or otherwise configured to generate a random alphanumeric sequence. The microprocessor may be part of or attached to any portion of the device.

In an example method of use, the system is used to document and verify mandatory rest intervals associated with commercial truck drivers. A commercial truck driver first secures the validation device 110 to his or her person, such as by wrapping the wristband around a wrist. The driver secures the wristband (with the GPS/time data logger) to his or her wrist with the frangible integrity seal immediately prior to initiating a period of monitored driving activity. The driver then records the device such as by placing his or her hand and wrist over the scanner 115 (FIG. 1) and scans the hand showing the identifying features of the wristband as well as unique or relatively unique anatomic characteristics, or biometrics. For example, the driver may scan the volar surface of his or her hand and the identifying features of the GPS/time data logger, as well as the frangible integrity seal to show that the seal is intact.

The scanner image obtained is then faxed (manually or automatically) or otherwise electronically transmitted to the data secure logging center 120 site that records the image with a time and date stamp, or some similar method or process of associating the image with a specific time and date of transmission. The logging center 120 may also transmit or transfer the data and scanned image at a specific time, for private and or public record (depending upon the specific requirements and the nature of authorized oversight). The driver may then immediately or shortly thereafter, receive confirmation of successful data transmission, such as via a mobile phone, smart watch, or other communications device that has wireless or wired data transmitting capability. This may be recorded within the personal data logger of the device or otherwise recorded.

When the driver elects to retire from a period of active monitoring, he or she then rescans the validation device 110, such as by scanning the same volar surface of their hand and the same identifying features of the GPS/time data logger, as well as the unbroken frangible integrity seal for transmission of the image to the logging center 120 for recording at a specific time. An electronic transmission of the GPS/time data log may then be concurrently sent to the driver, for his or her personal record keeping; as well as various other concerned and authorized parties. Once the driver receives confirmation of successful transmission of the time and location interval data, the driver is now free to break the frangible seal and retire from actively being monitored.

The stored and transmitted data at the logging center 120 may be automatically searched for compliance with mandatory intervals of stationary positioning (i.e. rest periods) as well as the locations of the monitored driver during the course of the interval. The information can be used for enforcement purposes as well as other purposes such as insurance compliance verification.

### Additional Embodiment

In another embodiment, the monitoring system and method includes two main components: 1) a personal time and position data logger device with associated unique encoded alphanumeric for the period of monitoring and the specific individual being monitored. The data logger device interfaces with a confirmatory biometric recording device, such as an anatomic feature imager/scanner (e.g. finger print or digital friction ridge pattern identifier) or physiologic biometric identifier (e.g. voice recognition software) at the beginning and end of the monitoring interval, and 2) an assessment device for assessing the sensorial arousal and cognitive state of an individual during a period of monitoring.

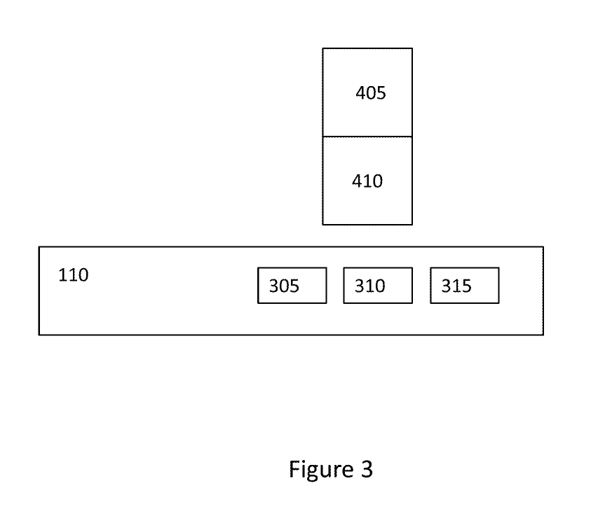

With reference to FIG. 1, a monitored individual 105 wears or otherwise dons a time and positional data logger device 110 on his or her person. The device 110 when secured to the person is configured to generate a unique and encoded alphanumeric sequence that is associated with a specific monitoring interval and may also include identifying information regarding the specific individual as well as the specific device. The encoded alphanumeric sequence is altered by any dissociation of the device 110 from the monitored person, such as a loosening of the device or when the individual doffs or removes the device from his or her person. In an embodiment shown in FIG. 3, the device 110 is a wrist band that includes an integral time and positional data logger 305 as well as microprocessor 310 and display 315.

When the band 110 is affixed to the person being monitored, the microprocessor generates a unique alphanumeric sequence that includes information specific or unique to the person, the specific device, as well as the date, the time, and the time period of monitoring. This information is displayed on the display 315, such as on the volar surface of the wrist. The information may be accessible in electronic form, possibly in the form of a bar encoded set or binary encoded sequence. When the wrist band is disconnected (or loosened sufficiently to be removed), the microprocessor alters the alphanumeric sequence unique for the period of monitoring in a manner to so indicate that the wrist band was or could have been dissociated from the monitored individual. A subsequent reattachment or securing of the wrist band results in the microprocessor 310 generating and displaying another unique or relatively unique alphanumeric code specific for a new monitoring interval.

In use, the monitored person might scan or photographically records his or her volar wrist and hand (along with the wristband) at the beginning of the monitoring interval. This can be done with or without a simultaneous or attached electronic or digitalized transmission of the information associated with the time and data logger as well as the unique alphanumeric sequence associated with the subject, the device, and the specific monitored interval. The information is transmitted to a distant site 120 (FIG. 1) for monitoring and/or retained in a secure location for storing such data. Prior to loosening or removing the device, the monitored person rescans or re-photographs his or her volar wrist and hand along with the device 110 and concurrently electronically transfer the data from the time and positional data logger device 110 to a distant monitoring site 120 or into a secure device of recording this information.

The logger device 110 can be fitted for a specific individual's anatomy (e.g. wrist circumference) such that once the device is secured to the individual, the device 110 cannot be removed without loosening or dissociation. If so removed or loosened, the microprocessor provokes an alteration in the alphanumeric sequence. The variety of unique biometrics associated with a digit, hand, or palm print are well established and various ways of recording and automating the identification of the individual associated with a palm or hand print exist and could be included in the wider scope of processing the data associated with distant site personnel monitoring. Alternatively, a unique or relatively unique physiologic characteristic of the individual, such as voice inflection pattern with or without a specific password or passwords, might be used to identify the specific individual being monitored.

Any of a variety of devices can be used to record/and or transmit the monitoring information of the device. There is now disclosed an example of a recording device 405 that can be used to record and/or transmit the previously described information and/or data via a personal phone (i.e. “smart phone”), microprocessor incorporated watch (e.g. “smart watch”), or other device with an integrated digital camera, scanner (e.g. digital scanner), or microphone (for voice recognition purposes).

As shown in FIG. 3, the recording device 405 includes a mount 410 that can be used to position the phone/camera at a preferred distance from the monitored person's wrist and the monitoring device 110 positioned on the wrist. The recording device 110 can be constructed of a lightweight and collapsible frame for ease of storage and transport and when deployed. The recording device 110 provides a reliable way for positioning the person's wrist and hand at a suitable distance for recording, while visually framing the material of interest, and excluding extraneous light from the photographed subject matter. The recording device can include hard wired connector(s) and/or a wireless data transmission connection to the phone from the monitoring device. Confirmation of a successful transmission of the data to a distant site via a call back or text message can be an added feature for the method described herein.

Alternatively, the volar surface of the wrist band and biometrics of the person's palm or digit(s) can be recorded and transmitted via a fax unit with computer recording of the time and positional data logger data, concurrently recorded, with or without transfer of this data to a distant site via the internet or other transmission means, such as phone lines. Another embodiment includes a digital friction ridge or finger print scanner, that is attached or integral to the recording device.

### Device for Assessing a Person's State of Sensorial Arousal and Cognitive State

There is now disclosed a device for assessing a monitored party's state of sensorial arousal and/or cognitive capacity or measuring a cognitive state or physiological parameter of the monitored party. Various sensorial stimuli (e.g. auditory or visual prompting) can be used periodically during the course of the monitoring period with a requirement for appropriate acknowledgement or response from the monitored subject. The requirement of acknowledgement provides a way of assessing the monitored person's sensorial arousal and cognitive functional state. Various stimuli can be presented to the person on a known or random schedule during the monitoring interval (e.g. shift, flight, drive, etc.) with a subsequent prescribed time limited interval, during which the monitored individual is expected to provide an acceptable or appropriate response. For example, a simple sequence of numbers and/or letters can be visually displayed or audibly articulated followed by a time limited interval in which the monitored person is required to verbally repeat or otherwise (e.g. key in) enter the sequence. In another embodiment, more cognitively demanding tasks might be required to fulfill the requirements of monitoring, such as calculations or recitations. Optionally included with the response is some means of assessing that the response was specific to the monitored individual, utilizing anatomic or physiologic characteristic(s) unique or relatively unique to the individual (e.g. finger print or voice inflection biometric features).

A failure of the person to properly respond to the sensorial prompting may result in various interventional consequences. The consequences may include but are not limited to the production of a local and/or distant monitoring site alarm, disabling of various equipment (e.g. accelerator on a truck), and/or enabling of safety response measures (e.g. braking of the vehicle and/or activation of the vehicles horn and emergency blinkers), etc. As indicated previously, digital friction ridge or voice recognition technology may be employed in the monitoring scheme to further ensure that biometric recognizable characteristics specific or relatively unique to the individual is recognized to reduce the risk of fraudulent or unauthorized responses.

The method may be realized in an application such as for a smart phone or smart watch. The application may include all or many of the components of this sensorial and cognitive monitoring method. The phone application generates the sensorial stimulus via auditory or visual prompting, with a requirement for appropriate responding from the monitored subject. Recording and/or responses or the absence of responses may be transmitted to a distant site or subsequently accessed from the phone. This information is transmitted along with the specific biometric and time and positional data recorded by the time and positional data logger wrist band.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A method of monitoring an individual, comprising:
securing a validation device to a monitored individual;
generating a unique identifying sequence upon securing the validation device to the individual;
making a first record of the alphanumeric sequence on the monitored individual showing an identifying feature of the validation device and a biometric associated with the individual;
electronically transmitting the first record to a logging center site that stores the record with a time and date stamp;
after an interval of time, making a second record of the validation device on the monitored individual showing the alphanumeric sequence, an identifying feature of the validation device and a biometrics;
electronically transmitting the second record to the logging center site that stores the record with a time and date stamp.

2. The method of claim 1, wherein the identifying sequence is an alphanumeric sequence.

3. The method of claim 1, wherein the identifying sequence is removed from the validation device upon removal of the validation device from the individual.

4. The method of claim 3, further comprising generating a new identifying sequence upon re-securing the validation device to the individual.

5. The method of claim 1, wherein the validation device is a wristband.

6. The method of claim 1, wherein making a record of the validation device comprises scanning the device.

7. The method of claim 1, further comprising receiving confirmation of successful data transmission.

8. The method of claim 1, wherein a mobile phone or smart watch is used to electronically transmit the first or second record.

9. The method of claim 1, wherein the biometric is at least one of a palm print, a digital print, and a voice recognized recording of the monitored party.

10. The method of claim 1, further comprising identifying or recording a measure of cognitive state or physiological parameter of the monitored party and electronically transmitting the measure of cognitive state to the logging center.

11. The method of claim 1, wherein the physiological parameter is a pulse rate of the monitored party.

12. A device for monitoringng an individual, comprising:
a location logging element that records a current location;
a time logging element that records a current time;
a personal monitoring attachment that attaches to an individual to be monitored, the personal monitoring attachment including the location logging element and the time logging element;
an identifying sequence generator that generates a unique identifying sequence when the personal monitoring attachment is attached to the individual, wherein the identifying sequence remains on display until the personal monitoring attachment is removed from the monitored individual.

13. The device of claim 12, wherein the time logging element indicates a time interval commenced with attaching the personal monitoring attachment to the individual and ending with the detachment of the personal monitoring attachment from the individual.

14. The device of claim 12, wherein the personal monitoring attachment is worn by the individual in a manner that is visibly juxtaposed to an anatomic feature characteristic of the individual.

15. The device of claim 14, wherein the personal monitoring attachment is worn around an individual's wrist.

16. The device of claim 12, wherein the identifying sequence is displayed in at least a partially encoded form.

17. The device of claim 12, further comprising a data transmitter.

18. The device of claim 17, wherein the data transmitter is a mobile.

19. The device of claim 12, further comprising a sensory stimulus generator that generates a sensory stimulus for response by the individual.

20. The device of claim 19, wherein the sensory stimulus is auditory.

21. The device of claim 19, wherein the sensory stimulus is visual.

22. The device of claim 19, wherein the sensory stimulus is tactile

23. The device of claim 19, wherein the stimulus is a sequence of alphanumeric characters to which the individual is required to respond to within a time limited interval.
